(19) United States
(12) Patent Application Publication
(10) Pub. No.: US 2010/0042000 A1
(43) Pub. Date: Feb. 18, 2010

(54) Method for correcting the image data that represent the blood flow

(76) Inventors: Thomas Schuhrke, Munich (DE); Guenter Meckes, Munich (DE)

Correspondence Address: ECKERT SEAMANS CHERIN & MELLOTT, LLC, U.S. STEEL TOWER, 600 GRANT STREET, PITTSBURGH, PA, 15219-2788, US

(21) Appl. No.: 12/462,045

(22) Filed: Jul. 28, 2009

(30) Foreign Application Priority Data
Jul. 28, 2008 (DE) 10 2008 040 807.7

(51) Int. Cl. A61B 6/00; G06K 9/00

(52) U.S. Cl. 600/476; 382/134

(57) ABSTRACT

A method for correcting the image data representing the blood flow for the evaluation and quantitative representation of the blood flow in a tissue or vascular region is based on the signal of a contrast agent injected into the blood. Several individual images of the signal emitted by the tissue or vascular region are recorded and stored at successive points in time. At least two individual images are correlated and a shift vector is generated based on the correlation. Thereafter, the image data of the individual images are shifted in relation to each other according to the shift vector.

### BACKGROUND OF THE INVENTION

The invention relates to a method for correcting the image data that represent the blood flow in a patient.

Several methods for observing and determining the blood flow in tissue and vascular regions are known in which a chromophore such as indocyanine green, for example, is applied. The fluorescent dye can be observed as it spreads in the tissue or along the blood vessels using a video camera. Depending on the area of application, the observation can be non-invasive or in the course of surgery, for example, via the camera of a surgical microscope.

Many methods are known, where only the relative distribution of the fluorescent dye in the tissue or in the blood vessels is examined qualitatively in order to draw conclusions concerning their blood flow. For example, conclusions are made about the blood flow and diagnoses are provided by watching an IR video recorded during surgery. It is also known to record an increase in the brightness of the fluorescence signal over time at all or at selected image points and in this manner record a time chart of the signal emitted by the fluorescent dye. The profile of the recorded formation plot provides the physician with qualitative information about potential vascular constrictions or other problems in the area of this image point. One example for this is provided in DE 101 20 980 A1.

However, a problem for such evaluations is that the recording unit or the object to be recorded may move during recording. In this case, the recorded video is shaky, the formation plot is unsteady and is not suited for further evaluations.

### SUMMARY OF THE INVENTION

The object forming the basis of the invention is to prepare recorded image data of a blood flow such that additional assistance can be derived from them for the medical professional providing treatment.

This objective, as well as other objectives which will become apparent from the discussion that follows, are achieved, according to the present invention, by the method and apparatus described below.

According to the invention, the image data that are obtained by observing the contrast agent flowing into the tissue or vascular area, whereby the signal emitted by said contrast agent is recorded via a series of images, preferably a video sequence and this series of images is split into individual images and/or stored, is corrected by correlating at least two individual images, determining a shift vector from this correlation and shifting the image data of at least one individual image according to the shift vector. Based on the correlation of two individual images, preferably recorded at points in time directly following each other or of images derived from them, the vector by which these individual images have shifted in their totality in relation to each other during recording can be easily determined. This offset can then be reversed by shifting the totality of the image data by this vector. In this manner, the respective image points of the object to be recorded are again laying on top of each other in the recorded individual images when the image series is viewed or evaluated. By correcting the image data of individual images using the shift vector, the negative influences that have been caused by moving the recording unit or the object during the recording can be overcome. This qualitatively allows data sets that are not optimal to be used for a reliable diagnosis. This fast and simple procedure for determining the shift vector is possible in particular, and can be done in real time, because the image contents of the images could not change much in the short period of time that passes between their recordings. In order to keep the method as simple as possible, preferably exactly one shift vector is determined for the correction of two individual images.

Preferably, the injected contrast agent is a fluorescent dye, such as indocyanine green, for example. However, other dyes known for perfusion diagnostics can be used as well. The excitation of the fluorescence for generating the signal to be obtained typically occurs via a near infrared light source. An infrared camera, which is often a CCD camera or a CMOS camera and which can be an autonomous medical device or can be integrated in a surgical microscope, is used for recording. The generation of the individual images of the signal that are to be recorded occurs either by splitting a continuous video into individual images or directly through storing recorded individual images in certain time sequences, which may be stored as a bitmap, for example.

In one preferred embodiment, the individual images are reduced to their essential components prior to the correlation. This simplifies the correlation method and provides a more reliable and unambiguous result of the correlation. By selecting only the significant components, all other differences between the images, which might lead to erroneous results, are neglected and are thus without meaning for the result. Only the significant features and their differences, i.e., essentially their offset, dominate the image contents and are visible at the correlation. One particularly advantageous method for reducing the image contents of the individual images to the significant image components is an edge detection method. With this method, the image content is reduced to the areas that exhibit big brightness transitions that follow, for example, the outlines of the blood vessels. This results in edge images that differ little from one individual image to the next in the image contents but differ because of movements during the recording, in the position of the image contents, i.e., in its offset. In general, a correlation of such edge images shows an unambiguous maximum corresponding in its offset in relation to the center with the shift vector between the correlated images.

In one advantageous embodiment, the shift vector is always determined between two in time directly successive individual images by correlating them or their edge images, respectively. Because the image content continuously changes during recording, it is difficult to determine the offset of two images to each other based on a correlation. No correlation is apparent if the image contents are very different and it is not possible to determine an offset. It is, therefore, advantageous to correlate only recordings that are very close in time because the image content between them has not yet changed significantly. The disadvantage of this method is that errors that result during the determination of the shift vector continue from one image to the next and add up.

For this reason, in another advantageous embodiment it is recommended to select a reference image with the offsets of the individual images being determined relative to them. In this manner, the offset of each individual image is always determined exclusively in relation to this reference image and each error that occurs in the process affects only the respective individual images.

Advantageously, the reference image is updated continuously by recording the image contents of several individual images into the reference image where the offset is determined and corrected in relation to the reference image. In other words, the reference image is continuously updated and supplemented with the data of the already corrected individual images. In this manner, all the image contents that have been contained in the individual images thus far are visible in the reference image, and the next individual image that is to be taken into account finds itself in the reference image with regard to its image contents and can therefore be well correlated such that its shift vector can be determined unambiguously.

In one particularly advantageous embodiment, an edge image that has been updated using the edge images of the corrected individual images is generated as the reference image. Thus, only the significant components of the individual images enter into the reference image resulting in a reference image that provides a good overview over all blood vessels visible in the individual images and thus ensures a very robust method for the determination of the shift vector.

Preferably, the reference image is developed by forming for every image point of the reference edge image the maximum of the reference edge image and the edge image of the current individual image taking into account the determined shift vector. In this manner all edges of blood vessels that indicated a strong contrast at some time during recording are visible in the reference edge image.

In one advantageous embodiment, the first reference image is determined automatically by correlating successive images. If the correlation coefficient determined in this manner exceeds a defined threshold value, then it can be assumed that initial clear contours of the contrast agent are formed that exceed the background noise and thus justify the determination of a first reference image.

For a full understanding of the present invention, reference should now be made to the following detailed description of the preferred embodiments of the invention as illustrated in the accompanying drawings.

### BRIEF DESCRIPTION OF THE DRAWINGS

FIG. 1 shows a schematic sequence of a method for representing the blood flow.

FIG. 2 shows an example of a profile of a brightness plot at one image point.

FIGS. 3a and b show an image of a blood vessel region and an associated edge image.

FIG. 4 shows the correlation of successive edge images and the shift vector derived from it.

FIGS. 5a and b show examples of blood vessel representations with and without movement compensation.

FIGS. 6a and b show examples of time offset representations generated using movement-compensated image data.

FIG. 7 shows schematically a surgical microscope for carrying out the method according to the invention.

### DESCRIPTION OF THE PREFERRED EMBODIMENTS

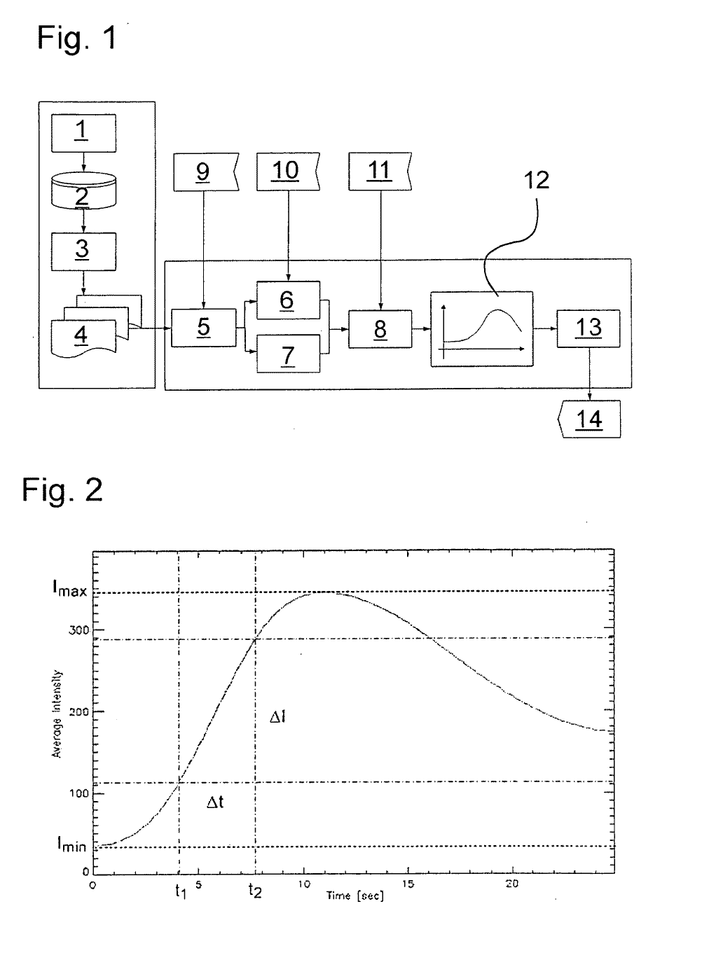

The preferred embodiments of the present invention will now be described with reference to FIGS. 1-7 of the drawings. Identical elements in the various figures are designated with the same reference numerals.

The complete system with the data flows and the individual processing steps is described in FIG. 1 and is used for representing and evaluating the blood flow. The data are recorded using a video camera 1 in the infrared range, which is arranged at the surgical microscope—not shown—or is a component thereof. The recorded infrared videos are stored in a data memory 2 and are split into individual images 4 using a video player 3. Alternatively, it is also possible to store the images of the video camera 1 directly as individual images 4. A frequency of five frames 4 per second proved to be useful. They are then corrected in a single image correction step 5. In the process, the corrections for the edge drop, the dark offset or of non-linearities of the video camera 1 are carried out taking into account the required correction data 9. The data of the corrected individual images 4 are than stored in the form of compressed binary data (e.g., Motion JPEG2000 Data (MJ2)) or in the form of non-compressed binary data (e.g., bitmap). In the form of non-compressed binary data, access times are shorter and the evaluation is faster.

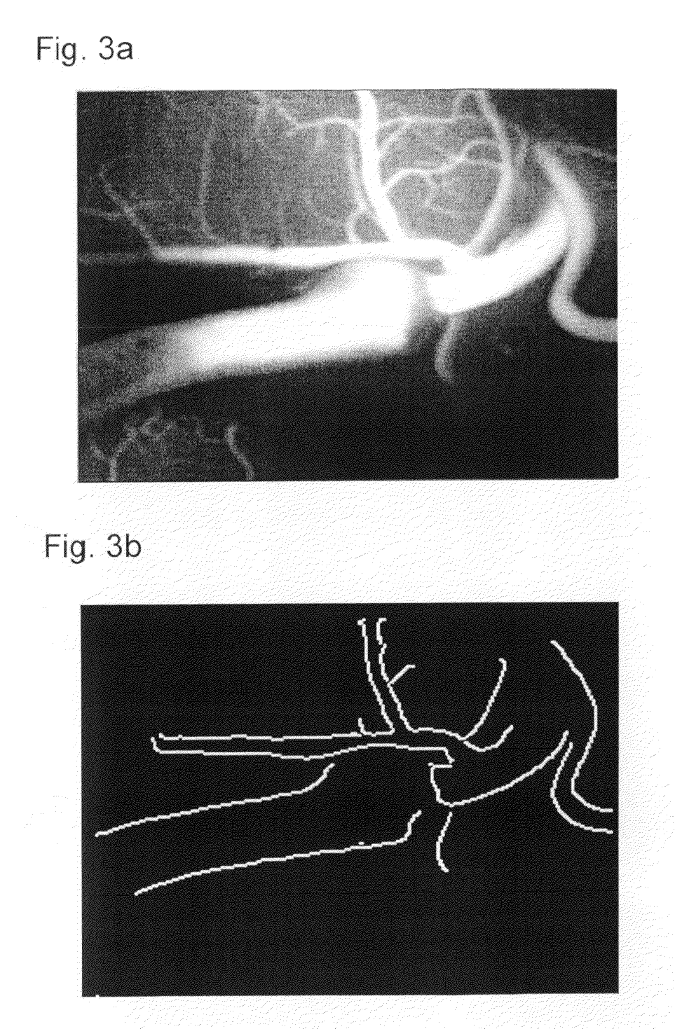

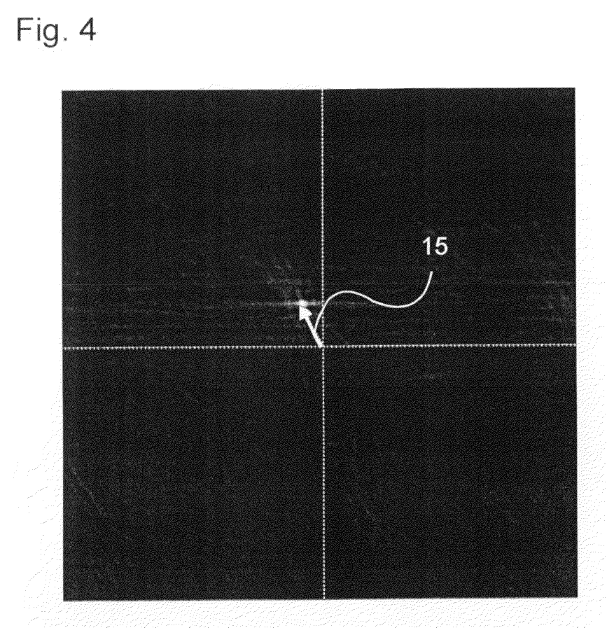

For the evaluation, the individual images 4 are transferred to the algorithms for the brightness correction 6 and movement compensation 7. For the brightness correction 6, for example, the different amplification factors that have been set at the video camera 1 are taken into account during the recording of the video in order to adapt the video camera 1 to the different fluorescence strengths of the tissue or vascular area to be recorded. They are documented during the recording as well, are stored in the data memory 2 as metadata 10 assigned to the video data and are computed with the individual images 4. During the movement compensation 7, the positions of the recorded individual images 4 are aligned. This is described below based on FIGS. 3 and 4.

The brightness determination 8 can be carried out following the corrections 6 and 7. For this purpose, first the position of the measurement range is determined in a measurement range determination 11. The measurement range within which the brightness shall be determined can be defined in a measurement range determination 11 via a measurement window or as a selection of specified measurement points. A clearly reliable result is achieved if the brightness for an image point is determined not only at this image point itself but in a measurement area around said image point, whereby an average is generated across all points of the measurement area. The result of the brightness determination 8 is a brightness plot 12 as a function of the time as can be seen in FIG. 2. This brightness plot 12 is computed for all or at least for a sufficiently large sample of image points.

In an evaluation 13, numerous other representations 14, comprising individual results as well, can be supplied from these brightness plots 12 and the individual images 4. They can then be represented on the screen together with the individual images 4.

In particular for surgeries, a movement compensation 7 is absolutely necessary in order to carry out this evaluation 13. The video camera 1 or the tissue or vascular area to be recorded may move during video recording. In such a case, the video is shaky, i.e., the same image point of the object is located at different positions in different individual images 4. However, when generating the brightness plot 12, the signal of an image point is observed and recorded across several individual images 4. If the position of the image point jumps, then jumps in the brightness plot 12 will result as well. It no longer progresses steadily. Thus, it is often not possible to derive maxima or threshold values or other quantities clearly from the brightness plot 12. It must first be ensured that when comparing the individual images 4 the same image point related to the object is always indeed viewed.

This is done by subjecting the individual images 4 prior to the evaluation 13 to a movement compensation 7, where their image points are assigned to the corresponding image points of the other individual images 4, i.e., where the individual images 4 are again aligned. The shift vector must be determined in order to align the individual offset images 4. In this case of the recording of the blood flow, it is assumed that all image areas of the individual image 4 are offset from each other by the same vector. The shift vector between two individual images 4 is determined by correlating these individual images 4. This provides a standardized similarity measure, the correlation coefficient. To perform the correlation more efficiently and more reliably, the individual images 4 are first reduced to their significant features. This makes the process more reliable.

The edge detection method can be used for this purpose. The so-called Canny edge detector has proven to be particularly advantageous for this application. Based on this method, in which an edge detection algorithm is applied to individual images 4, an edge image is generated from an individual image 4 of the video recording by isolating the progression of strong contrasts and thus the significant structures in the image. One example for this can be seen in FIGS. 3a and 3b. FIG. 3a shows a section of an individual image 4, in which a blood vessel region is represented as a grayscale image. The center of the image shows two light blood vessel sections with a strong flow of a fluorescence agent that are clearly delineated from the dark background. FIG. 3b shows an edge image of this section from an individual image 4. The contours of the two light blood vessel sections are clearly visible.

The correlation of the edge images of two individual images is carried out in the frequency domain. To this end, the two edge images are Fourier transformed, the results multiplied with each other and the product reverse-transformed. The position of the maximum is determined in the absolute amount of the reverse-transformed. One example for this is shown in FIG. 4. Here, the maximum of the correlation can be seen as a light spot slightly offset from the center in the upper left sector. The deviation of the maximum position from the center corresponds directly to the drawn-in shift vector 15. If this vector is applied to one of individual images 4, then the individual images 4 will be aligned, the positions of the image points will be aligned and an error-free unambiguous evaluation will be possible.

Fundamentally, however, one additional problem must be taken into account in the movement compensation 7 of recordings showing the blood flow. Because the image information in the individual images 4 changes constantly, the correlation of individual images 4 or of the edge images will not always lead to such an unambiguous result as can be seen in FIG. 4. In the worst case, the image content of two individual images 4 is entirely different, and the correlations exhibits no pronounced maximum. To overcome this problem, only directly successive individual images 4 are used for the correlation at all times. This ensures that the recorded image content did not yet change too much. Each following individual image 4 is correlated with the respective current movement-compensated individual image 4 or its edge image. An additional possibility consists in generating an output signal that serves as a reference image for all following individual images 4. For this purpose, an initial reference image is selected from among the individual images 4. The image on which clear structures can be recognized, for example, on which the standard deviation exceeds a defined threshold value, can serve as an initial reference image. Based on this initial reference image, the correlation with a following individual image 4 is used to determine the degree of offset in relation to the initial reference image. The offset is reversed such that the initial reference image and the individual image 4 are aligned. Thereafter, a new reference image is formed by combining the edge information of the previous reference image and the corrected individual image 4 or its edge image. This is done for each image point of the new reference image by entering the greater value of the two individual images 4 that have been placed on top of each other. This method continues iteratively. A reference image is thus created in which in the end all edges of blood vessels that have at some time during the video recording shown a strong contrast and that are present in the corrected individual images are visible. This contributes significantly to the robustness of the method because this reference image is correlated with all individual images 4 resulting in a pronounced maximum. In addition, the reference image that was established in the end can also be used for additional evaluations and representations via the position of the blood vessels.

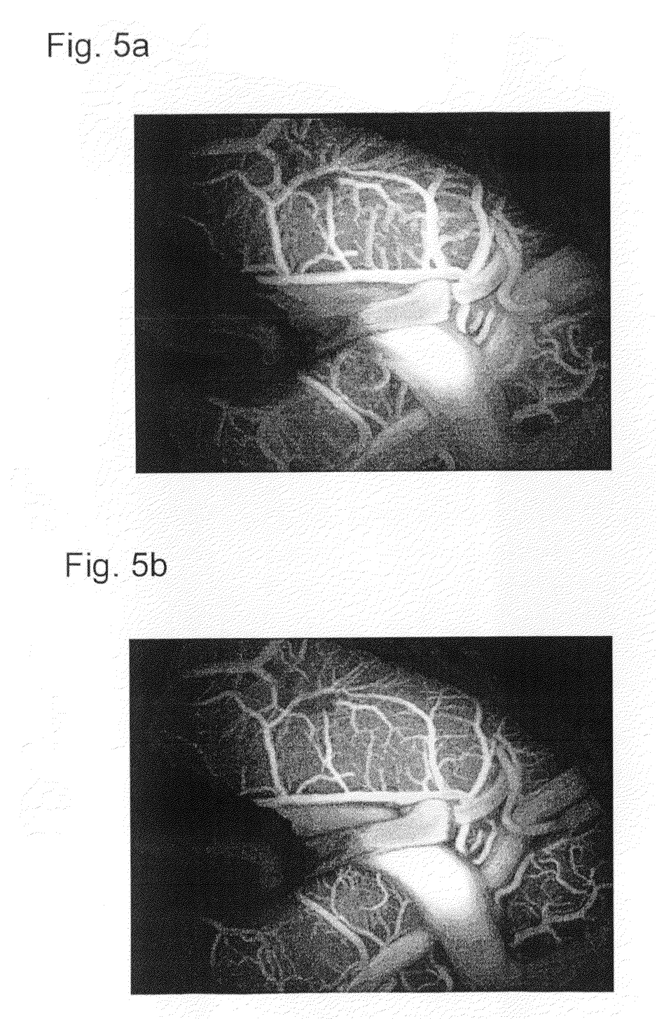

One example for an evaluation that can be performed reliably only after a concluded movement compensation 7 is a so-called blood vessel representation, where all vessels and all tissues in which fluorescent agents have flowed through appear light and thus provide an overview over the position and the progress of the blood vessels. This representation is generated by presenting the difference between the maximum and minimum brightness value for each image point of the superimposed individual images 4. With this maximum brightness for each image point, one obtains a relative, quantitative quantity for the blood flow at all positions. This enables the physician to recognize defects. Examples for blood vessel representations can be seen in FIGS. 5a and 5b. FIG. 5a shows a blood vessel representation that has been generated without movement compensation 7, while FIG. 5b shows an example with movement compensation 7. Clearly recognizable is the significantly better sharpness of the contours in FIG. 5b with movement compensation. In FIG. 5a, on the other hand, the blood vessels can be seen with extremely blurry edges such that an exact localization appears impossible, especially for the finer blood vessels.

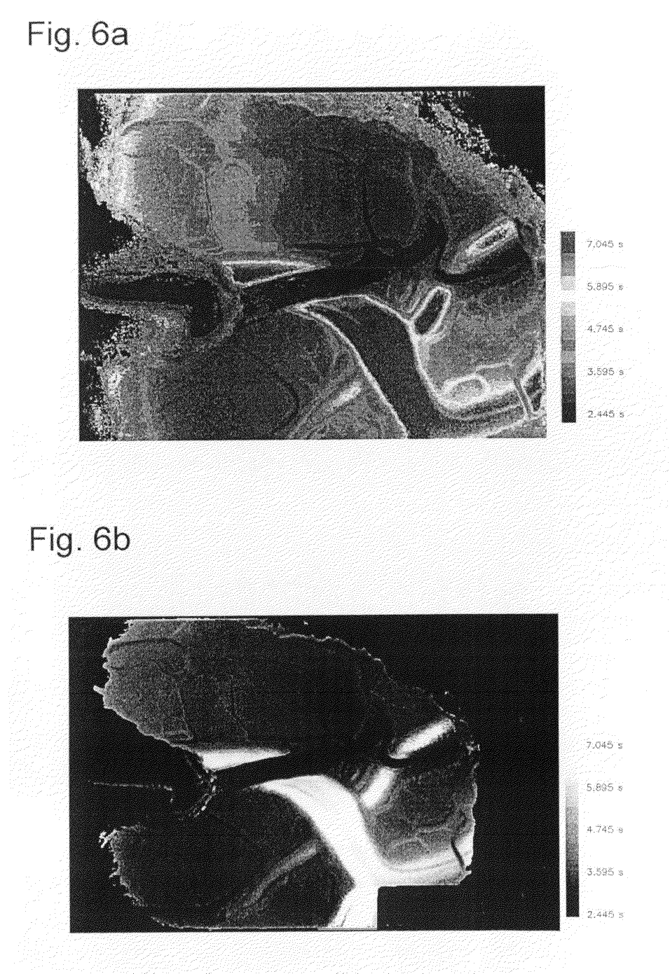

An additional representation 14 for which a movement compensations 7 is an important prerequisite, is shown in FIGS. 6a and b. It provides a false color image representing the time offset. FIG. 6a shows the onset time of the blood flow in a color representation transferred into grayscale, whereby the bars on the right side show the false color scale, i.e., the relationship between the selected colors and the respective elapsed time. The false color scale is selected such that an intuitive correlation to known anatomic terms exists. Accordingly, red is selected for an earlier point in time, i.e., a small time offset, in order to emphasize the arterial character, and blue for a later point in time, i.e., a greater time offset, to accent the venous character. In FIG. 6a, the color scale thus transitions from red (here at about 2.5 sec) to green (here at about 5 sec) and finally to blue (here at about 7 sec). In this manner, the physician receives a quick overview of the time when the blood arrived at which position of the blood vessel. Thus, using the time offset, information about the inflow and outflow of the blood in the blood vessels or in the tissue is made transparent. Because the conversion of the false color image into grayscale does not permit an unambiguous assignment of the colors, a similar representation 14 of a time offset in place of a false color image has been implemented as a grayscale image with a grayscale for black and white representations as are necessary here, for example, or also for black-and-white screens. This can be seen in FIG. 6b. Here, blood vessels into which the blood with the fluorescent dye flows immediately are shown dark while the blood vessels that the blood reaches later are shown very light. However, the grayscale representation has less information contents compared to the false color representation. Other types of representation such as a three-dimensional representation, for example, where the third dimension is the time, are conceivable as well.

To generate the representation 14, a brightness plot 12 is computed for each image point based on all individual images 4 of the video. Then the point in time t₁ at which the brightness plot 12 has exceeded a certain threshold value I(t₁) is determined for each image point. The threshold value is defined as I(t₁)=Iₘᵢₙ+0.2×(Iₘₐₓ−Iₘᵢₙ). This point in time is converted to the respective color, grayscale or height and entered into the time offset representation, Iₘₐₓ and Iₘᵢₙ must be determined by comparing the recorded data of several individual images 4 in order to determine the threshold value I(t₁). To obtain an unambiguous result, it is extremely important to carry out a movement compensation 7 first. Without movement compensation 7, the brightness plot 12 is not steady such that several Iₘₐₓ and Iₘᵢₙ could arise in each brightness plot 12. The same applies to the brightness correction 6. Without a brightness correction 6, a steady plot would also not arise for recording devices where the recording conditions may change during the recording of the individual images 4 and where the changes affect the brightness of the individually recorded images 4. Changes in the recording conditions are necessary, whenever a greater contrast range is to be covered.

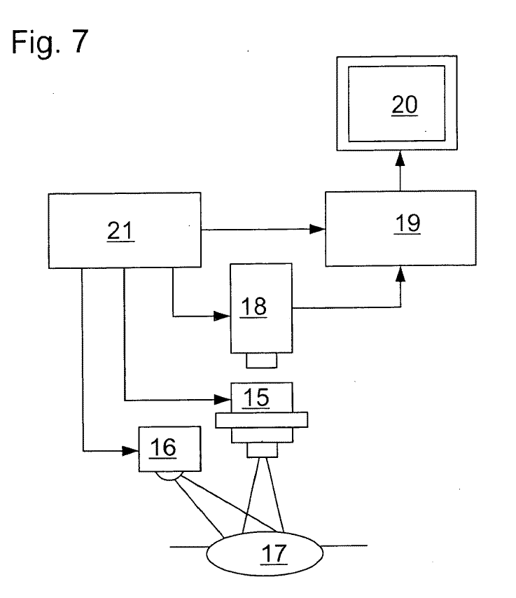

FIG. 7 shows schematically the essential components of a surgical microscope that can be used to apply the method according to the invention. The optics 16 of a surgical microscope reproduces an object 18, for example the head of a patient that is to be treated during surgery and is illuminated by a light source 17 of the surgical microscope, in a camera 19. The camera 19 can also be a component of the surgical microscope. The image data recorded by the camera 19 are transferred to a computer unit 20 where they are evaluated. Medical quantities derived at the evaluation are then represented on the screen 21, potentially together with the recorded image. Similar to the computer unit 20, the screen 21 can be a component of the central surgical control but can also be a component of the surgical microscope. A control unit 22 controls the brightness of the light source 17 as well as the magnification factor and the aperture of the optics 16 and the amplification factor of the camera 19. In addition, the control unit 22 generates metadata that provide information about changes in the recording conditions that occur as soon as the control unit 22 adjusts one of the quantities that is to be controlled. These metadata are transferred from the control unit 22 to the computer unit 20, where they are assigned to the image data that have been provided to the computer unit 20 by the camera 19. Metadata and image data are stored, at least temporarily, by the computer unit 20 and are evaluated according to the method according to the invention. During the evaluation, the metadata are included with the image data. The results of the evaluation according to the invention are then displayed on the display unit 21, possibly together with the image data.

There has thus been shown and described a novel method and apparatus for the correcting the image data that represent the blood flow which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. A method for correcting the image data representing blood flow for the evaluation and quantitative representation of the blood flow in a tissue or vascular region based on the signal of a contrast agent injected into the blood, said method comprising the steps of:
recording and storing, at successive intervals of time, several individual images of the signal emitted by the tissue or vascular region,
correlating at least two individual images or images derived from the stored images,
generating a shift vector based on the correlation, and
shifting the image data of the individual images in relation to each other according to the shift vector.

2. A method for correcting the image data representing blood flow as set forth in claim 1, wherein prior to the correlation, the individual images are converted to edge images using an edge detector.

3. A method for correcting the image data representing blood flow as set forth in claim 1, wherein individual images in close time proximity are correlated.

4. A method for correcting the image data representing blood flow as set forth in claim 1, wherein individual images are each correlated with a reference image.

5. A method for correcting the image data representing blood flow as set forth in claim 4, wherein the reference image is generated using image data of several corrected individual images.

6. A method for correcting the image data representing blood flow as set forth in claim 4, wherein the reference image is an edge image that is being supplemented with the edge image of prior individual images.

7. A method for correcting the image data representing blood flow as set forth in claim 4, wherein an individual image is selected as the initial reference image, where the correlation coefficient between said individual image and the directly following individual image exceeds a defined threshold value.

8. A surgical microscope for recording a fluorescence radiation of a contrast agent comprising a camera for recording an image sequence of an object, wherein the camera is connected to a computer unit for deriving medical quantities from an image sequence of medical image data or individual images of the image sequence, and with a display for displaying the image data to be evaluated, the improvement wherein the computer unit operates in accordance with a program for carrying out the method as set forth in claim 1.

9. An analysis system of a surgical microscope for recording a fluorescence radiation of a contrast agent, comprising a computer unit that operates in accordance with a program for performing the method as set forth in claim 1.
